(19) United States
(12) Patent Application Publication
(10) Pub. No.: US 2010/0274277 A1
(43) Pub. Date: Oct. 28, 2010

(54) EMBOLIC PROTECTION DEVICE WITH MAXIMIZED FLOW-THROUGH

(75) Inventor: Elizabeth A. Eaton, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/430,403

(22) Filed: Apr. 27, 2009

(51) Int. Cl. A61F 2/01

(52) U.S. Cl. 606/200

(57) ABSTRACT

An embolic protection device for capturing emboli during treatment of a lesion in a blood vessel is presented. This embolic protection device generally comprises a plurality of struts having a predetermined shape and being configured to move between an expanded state for engagement with the blood vessel and a collapsed state for filter retrieval and delivery and a filter portion circumferentially attached to the struts having a proximal end and a distal end; the filter portion extending freely from the proximal end to a closed distal end. The filter portion forms at least one annulus chamber in the expanded state with the closed distal end of each chamber being not coincident to the center longitudinal axis of the blood vessel in order capture emboli in the chambers and to reduce any overall restriction of blood flow through the filter portion.

### FIELD

This invention relates generally to medical devices. More particularly, the present invention relates to embolic protection devices and methods for capturing emboli within a body lumen.

### BACKGROUND

Due to the continuing advance of medical techniques, interventional procedures are becoming more commonly used to actively treat stenosis, occlusions, lesions, or other defects within a patient's body vessel. Often the region to be treated is located in a coronary, carotid or cerebral artery. One example of a procedure for treating an occluded or stenosed body vessel is angioplasty. During angioplasty, an inflatable balloon is introduced into the occluded region. The balloon is inflated, pushing against the plaque or other material in the stenosed region. As the balloon presses against the material, portions of the material may inadvertently break free from the plaque deposit. These emboli may travel along the vessel and become trapped in smaller body vessels, which could result in restricting the blood flow to a vital organ, such as the brain.

To prevent the risk of damage from emboli, many devices have been used to restrict the flow of emboli downstream from a stenosed region. One such method includes inserting a balloon that may be expanded to occlude the flow of blood through the artery downstream of the stenosed region. An aspirating catheter positioned between the balloon and stenosed region may be used to remove any emboli resulting from the treatment. However, the use of this procedure is limited to very short intervals of time because the expanded balloon will completely block or occlude the blood flow through the vessel.

As an alternative to occluding flow through a body vessel, various filtering devices have been used. Such devices typically have elements incorporating interlocking leg segments or a woven mesh that can capture embolic material, but allow blood cells to flow between the elements. Capturing the emboli in the filter device prevents the material from becoming lodged downstream in a smaller body vessel. The filter may subsequently be removed from the body vessel along with the embolic material after the procedure has been performed and the risk from emboli has diminished.

However, various issues exist with the design, manufacturing, and use of existing filtering devices. Often it is desirable to deploy filter devices from the proximal side of the stenosed region. Therefore, the profile of the filtering device should be smaller than the opening through the stenosed region. In addition, the filter portion may become clogged or occluded during treatment, thereby, reducing the blood flow through the body vessel. Moreover, many filtering devices are difficult to collapse and retrieve from the body vessel after the need for such a device no longer exists.

Accordingly, there is a need to provide improved devices and methods for capturing emboli within a body vessel, including providing distal protection during a procedure that has the potential to produce emboli without relatively restricting blood flow through the vessel and with relatively easy retrievability of the device.

### SUMMARY

The present invention generally provides an embolic protection device that minimizes restricted flow when deployed within the vasculature of a patient and that is relatively easy to retrieve after the majority of the risk of generating new blood clots and thrombi within the vasculature has passed. The embolic protection device includes a set of wires arranged as a plurality of struts. These struts are coupled together at their distal ends as well as to the distal end of a core wire. Another section of the wires spirals around the core wire to define a hollow channel in which the core wire can reciprocate. Thus, pulling or pushing a proximal end of the core wire relative to the spiraled section expands or contracts the struts.

A filter portion is attached to the struts for capturing emboli when the struts are in an expanded configuration. The filter portion forms at least one annulus chamber in the expanded state with the closed distal end of the chamber being not coincident with the longitudinal central axis X. The annulus chamber may be concentric about or off-center from the longitudinal central axis. During treatment, the emboli are forced by the blood flow to move into the most distal part of the annulus chamber where they are caught or held.

The filter portion, struts, and deployment mechanism are all one integral unit having a small cross sectional profile when the embolic protection device is in a collapsed configuration. Thus, during delivery of the device, this small profile enables the device to pass by a lesion without inadvertently dislodging excessive material from the lesion site.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1A is a schematic representation of the velocity profile for blood flow viewed through a cross section of a blood vessel;

FIG. 1B is a schematic representation of the velocity profile for the blood flow of FIG. 1A viewed end-on;

FIG. 2A is a side-view of an embolic protection device in a deployed state made in accordance with the teachings of the present invention;

FIG. 2B is a side-view of an embolic protection device in a deployed state made according to another aspect of the present invention;

FIG. 2C is a schematic representation of the embolic protection device of FIG. 2A in a top-down view further depicting a concentric annulus;

FIG. 2D is a schematic representation of the embolic protection device of FIG. 2A in a side-view depicting the concentric annulus;

FIG. 2E is a side-view of the embolic protection device of FIG. 2A shown in a collapsed state; and

FIG. 2F is a side-view of the embolic protection device of FIG. 2B shown in a collapsed state.

FIG. 3A is a sectional view of a body vessel or lumen illustrating insertion of the embolic protection device of FIG. 2A in a collapsed state;

FIG. 3B is a sectional view of the body vessel illustrating the embolic protection device of FIG. 2A in a fully deployed state;

FIG. 3C is a sectional view of the body vessel illustrating removal of the embolic protection device of FIG. 2A from the vessel;

FIG. 4A is a side view of an embolic protection assembly for capturing emboli during treatment in accordance with one embodiment of the present invention;

FIG. 4B is an exploded side view of the assembly of FIG. 4A; and

FIG. 5 is a flow chart of one method for providing embolic protection during treatment of a stenotic lesion in a blood vessel.

### DETAILED DESCRIPTION

The following description is merely exemplary in nature and is in no way intended to limit the present disclosure or its application or uses. It should be understood that throughout the description and drawings, corresponding reference numerals indicate like or corresponding parts and features.

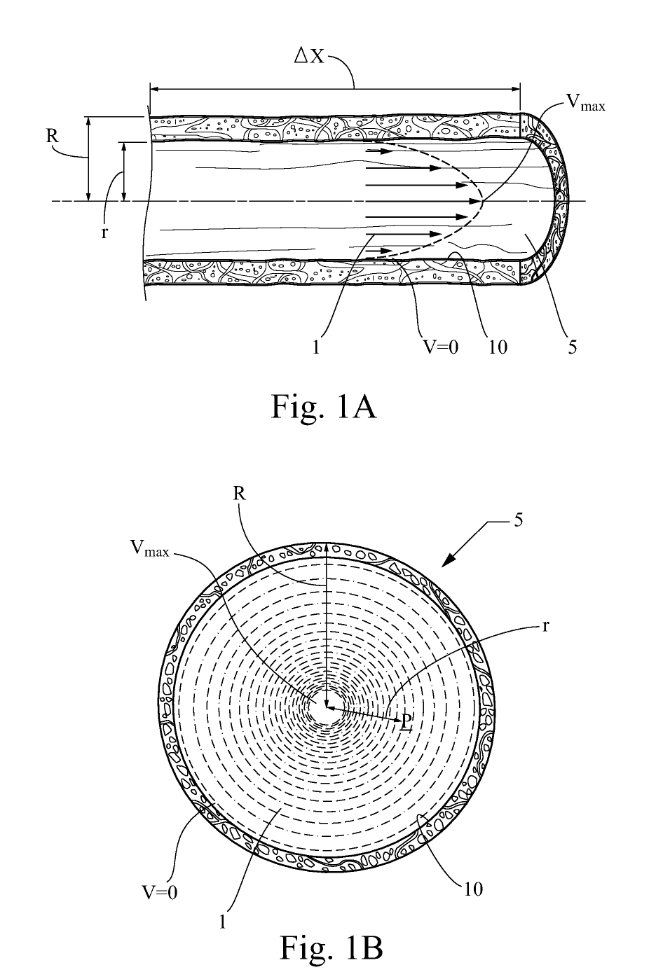

Even though arterial flow is always pulsatile, more or less so according to the distance from the heart, and the occurrence of some degree of turbulence is likely, especially in the region of a stenotic lesion, laminar flow as shown in FIGS. 1A and 1B, is the normal regime through which blood 1 flow may be modeled throughout most of the circulatory system. Laminar flow is characterized by concentric layers of blood 1 moving in parallel down the length of a blood vessel 5. The maximum velocity (Vₘₐₓ) for blood 1 flow is found near the center of the vessel 5, while the lowest velocity (V=0) is found proximate to the vessel wall 10. Under steady flow conditions, the flow profile for blood 1 flow through a blood vessel 5 can be approximated as parabolic in nature as shown in FIGS. 1A and 1B. The orderly movement of adjacent layers of blood 1 flow through a vessel 5 helps to reduce energy losses in the flowing blood 1 by minimizing viscous interactions between the adjacent layers of blood 1 and the wall 10 of the blood vessel 5. This type of blood 1 flow, as well as the effect of vasodilation and arterial occlusion, is adequately described by Poiseuille's Law.

The maximum velocity (Vₘₐₓ) for the blood 1 flow may be derived according to Equation 1, where η is the viscosity of the blood 1, the variable R is the radius of the blood vessel 5, and the ratio ΔP/Δx is the pressure gradient along a predetermined length of the blood vessel 5. The velocity profile for any point P in the blood vessel 5, may then be determined according to Equation 2, where the distance r between the point P and the centerline of the blood vessel 5 is known.

$$\begin{matrix}{v}_{m} = \frac{1 Δ P}{4 η Δ x} {R}^{2} & Eq . 1 \\ v ( r ) = {v}_{m} ( 1 - \frac{{r}^{2}}{{R}^{2}} ) & Eq . 2\end{matrix}$$

Maintaining normal flow conditions in a blood vessel 5 is difficult to accomplish when using a conventional embolic protection device having a centrally located filter mesh. Capturing of emboli by this filter mesh results in the mesh becoming plugged or at the very least; restricting the flow of blood 1 through the center portion of the filter where the velocity of blood 1 flow usually is at a maximum. The present invention generally provides an embolic protection or capture device that reduces restricted flow when deployed within the vasculature of a patient and that is relatively easy to retrieve after the risk of releasing blood clots, thrombi, and other emboli within the vasculature has passed. Embodiments of the present invention generally provide an embolic protection device comprising a plurality of struts having first ends attached together along a longitudinal axis and a filter portion that is circumferentially attached to the struts. When deployed in a blood vessel 5, the filter portion opens to an expanded state of the device allowing blood 1 to flow there through for capturing emboli. The struts of the embolic protection device allow for relatively easy removal from the body vessel 5. This may be accomplished by distally threading a catheter over the struts until the filter is collapsed within the catheter.

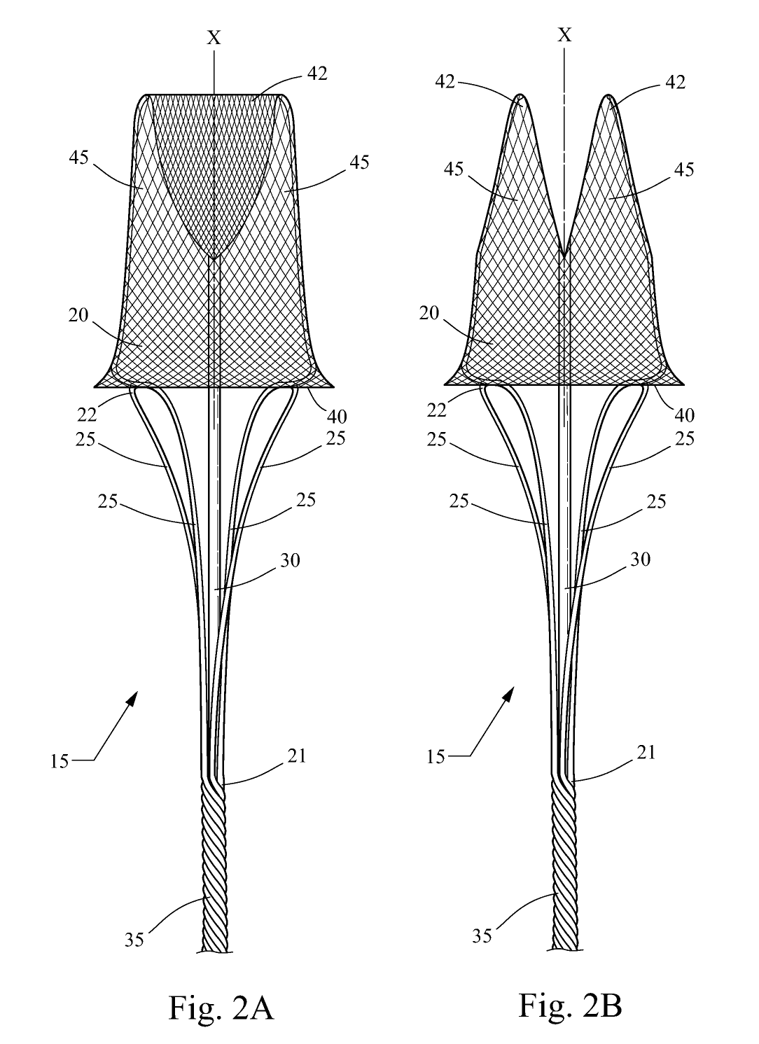

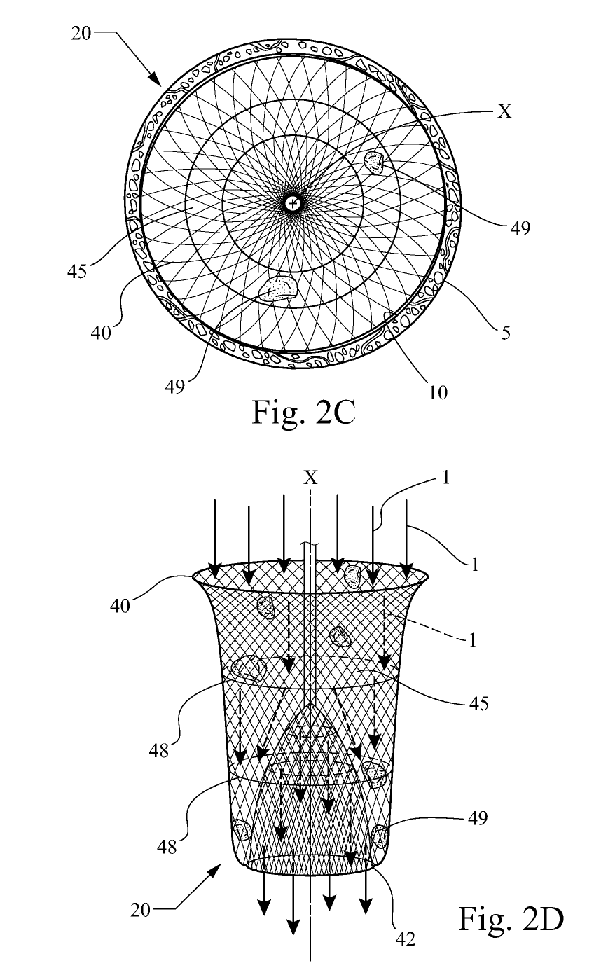

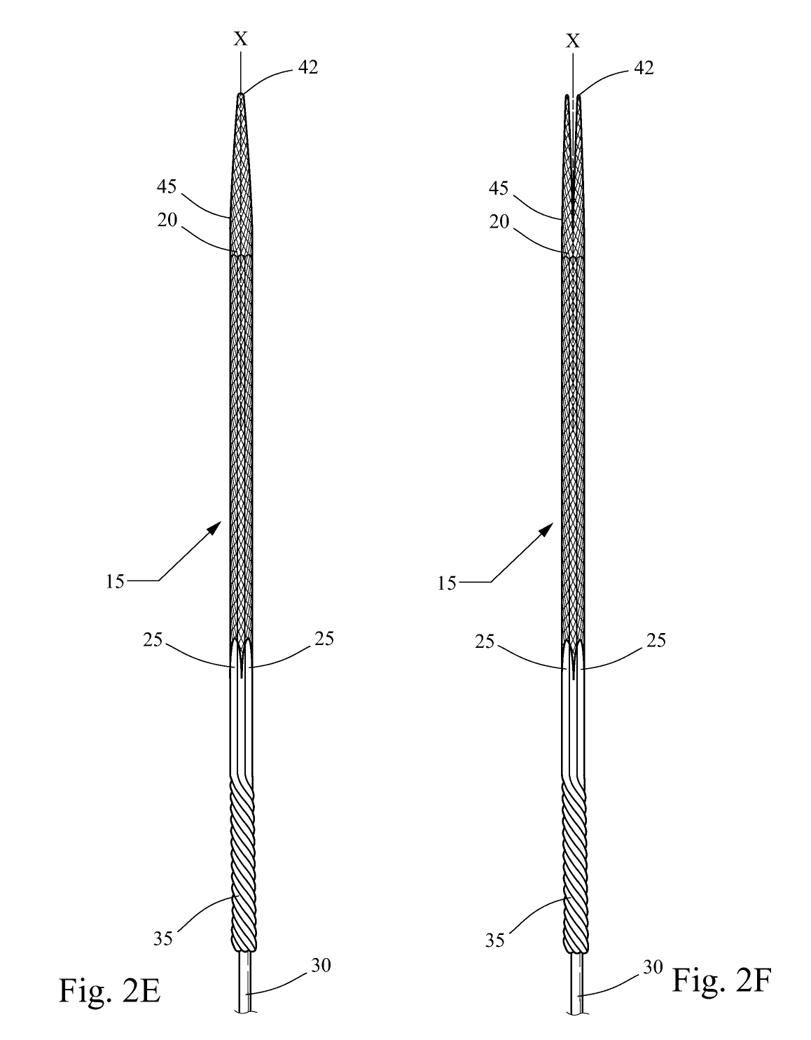

Referring to FIGS. 2A and 2B, the embolic protection device 15 made according to various embodiments of the present invention is shown to comprise a filter portion 20 and a plurality of struts 25 each having a predetermined shape and a proximal end 21 attached together at a position that is central along a longitudinal axis X. The struts 25 are defined by a section of a set of wires arranged as so that they extend longitudinally from the proximal end 21 of the embolic protection device 15 to a distal end 22. The set of wires is twisted or spiraled to define a spiraled section 35 with a hollow channel through which a core wire 30 is slideably received and extends along the longitudinal axis X of the device 15. According to one aspect of the present invention, the core wire 30 may be attached to the distal end 22 of the struts 25. The proximal end of the core wire 30 extends beyond the proximal end of the spiraled portion 35 of struts 25. The core wire 30 may be attached or coupled to the struts 25 by solder or by being embedded in a plastic material.

The lip of the filter portion 40 is attached to the struts 25 at attachment points that may be proximal to the distal end 22 of the struts 25 to define an opening into which clots or emboli flow when the filter is deployed in the vasculature. The attachment points may be attached using glue or solder or any other biocompatible attachment mechanism. When in the expanded configuration, the struts 25 extend longitudinally and curve outwardly between the proximal end 21 and the distal end 22. The attachment points are typically located on the struts 25 approximately where each strut 25 achieves its maximum diameter when expanded so that blood 1 flows through the filter portion 20 and not around it.

Since the core wire 30 may be attached at the distal end 22 of the struts 25 and is able to reciprocate within the hollow channel of the spiraled section 35, grasping the proximal end of the core wire 30 and pulling it relative to the proximal end of the spiraled section 35, causes the struts 25 to expand and hence also the filter portion 20. Conversely, pushing the core wire 30 relative to the spiraled section 35 collapses the struts 25 and filter portion 20 for delivery or retrieval of the embolic protection device 15. This feature allows a catheter to ride over the spiraled section 35 and the struts 25 for relatively easy collapse and retrieval of the device 15. As shown in FIGS. 2A and 2B, four wires define the struts 25 and the spiraled section 35. However, depending on the application, less than or more than four struts may be employed.

The filter portion 20 extends freely from the lip 40 at its proximal end to a closed distal end 42. The filter portion 20 forms at least one annulus chamber 45 in the expanded state with the closed distal end 45 being not coincident with the longitudinal central axis X. Referring to FIG. 2A, the filter portion 20 preferably has an annulus chamber that is concentric with or about the longitudinal central axis X. During treatment, the emboli will be forced by the blood 1 flow to move into the most distal part of the filter portion 20 where they will be caught or held. The most distal part of the filter portion 20 is the annulus chamber 45, which is concentric with but not coincident to the longitudinal axis X of the device 15. Preferably, the longitudinal axis X of the device 15 is positioned proximate to the center axis of a blood vessel.

Referring now to FIGS. 2C and 2D, further depiction of the filter portion 20 extending freely from a lip 40 at its proximal end and forming an annular chamber 45 closed at its distal end 42, the annular chamber 45 being concentric with but not coincident to the longitudinal central axis X of the blood vessel 5. Since the filter portion 20 is deployed in the blood vessel 5 at a point that is beyond a lesion, the geometry of the vessel 5 may be typically approximated as being a series of circles 48 when viewed as a series of radial slices taken perpendicular to the vessel. In this case, the forces acting against the radial expansion of the filter 20 structure are found to be relatively close to uniform within each radial slice. Since the velocity of the blood 1 flow is most likely to be at a maximum near the center of a blood vessel 5 and approximately zero at the wall 10 of the blood vessel 5, the annular chamber 45 being located concentric with but not coincident to the longitudinal axis X resides closer to the wall 10 of the blood vessel 5 where the blood 1 flow is reduced. Emboli 49 becoming caught and held in the annular chamber 45 will exhibit less of an effect on the overall blood 1 flow than if the emboli were caught in the part of the filter portion 20 that is proximate to the central axis of the blood vessel 5 were the blood 1 flow approaches its maximum velocity. In other words, capturing emboli in the off-center annular chamber 45 reduces the restriction of blood 1 flow during treatment.

The shape of the annulus chamber 45 as depicted in FIGS. 2C and 2D only represents one aspect of the present invention. One skilled-in-the-art will understand that the shape of the annulus chamber 45 can be varied without departing from the scope of the invention. For example, the closed distal end of the annulus chamber 45 may be triangular or pointed as shown in FIG. 2D, rounded, square (i.e., flat), or any other desired shape or geometry.

In FIG. 2B, a filter portion 20 made according to another aspect of the present invention is shown in its expanded state to form multiple annulus chambers 45. During treatment, the emboli are forced by the blood flow to move into the most distal part of the filter portion 20 where they will be caught or held. In this case, the multiple annulus chambers 45 each have a closed distal end 42 that is not coincident with, but rather off-center from the longitudinal central axis X of the device 15. Preferably, the longitudinal axis X of the device 15 is positioned proximate to the center axis of a blood vessel 5

FIGS. 2E and 2F illustrate the device 15 in its collapsed or closed state in accordance with various embodiments of the present invention. As shown, the device 15 has a reduced diameter, occupying a cross-sectional profile less than the outer diameter of the device 15 in the corresponding expanded state (see FIGS. 2A and 2B). The struts 25 are generally straight and the filter portion 20 is collapsed about a portion of the struts 25. The part of the filter portion 20 extending beyond the distal end of the struts 25 may be folded back over the struts 25 for delivery of the device 15.

The struts 25 may be formed from any suitable material such as a superelastic material, Nitinol, stainless steel wire, cobalt-chromium-nickel-molybdenum-iron alloy, or cobalt-chrome alloy. It is understood that in some implementations the struts 25 may be formed of any other suitable material known to one skilled-in-the-art that will result in a self-opening or self-expanding structure, such as shape memory alloys. Shape memory alloys have the desirable property of becoming rigid, e.g., returning to a “remembered state”, when heated above a preset transition temperature. A shape memory alloy suitable for the present invention is a Ni—Ti alloy or Nitinol. When this material is heated above its transition temperature, the material undergoes a phase transformation from martensite to austenite, such that the material returns to its remembered state. The transition temperature is dependent on the relative proportions of the alloying elements Ni and Ti and the optional inclusion of alloying additives.

In one embodiment, the struts 25 are made from Nitinol with a transition temperature that is slightly below normal body temperature of humans (that is, about 98.6° F). Thus, when the embolic protection device 15 is deployed in a blood vessel 5 and exposed to normal body temperature, the alloy of the struts 25 will transform to austenite (i.e., the remembered state), which for certain implementations is the expanded configuration when the embolic protection device 15 is deployed in the body vessel 5. To remove the embolic protection device 15, the struts 25 may be cooled, for example, with a refrigerated saline solution, to transform the material to martensite, which is more ductile than austenite, making the struts 25 more malleable, and hence more easily collapsible by pushing the core wire 30 relative to the spiraled section 35 and then pulling the device 15 into a lumen of a catheter for removal.

In another embodiment, the struts 25 may be self-closing or self-collapsing. In this case, the struts 25 may be made from Nitinol with a transition temperature that is above normal human body temperature. Thus, when the embolic protection device 15 is deployed in a blood vessel 5 and exposed to normal body temperature, the struts 25 are in the martensitic state so that they are sufficiently ductile to bend or form the device 15 into an expanded configuration. To remove the embolic protection device 15, it is heated, for example, with a warm saline solution, to transform the alloy to austenite so that the struts 25 become rigid and return to the remembered state, i.e., the collapsed configuration

The filter portion 20 may be formed from any suitable material to be used for capturing emboli 49 from a stenotic lesion during treatment thereof while allowing blood 1 to flow through it. In one embodiment, the filter portion 20 may be made partially of connective tissue material for capturing emboli 49. The connective tissue may include extracellular matrix (ECM), which is a complex structural entity surrounding and supporting cells that are found within mammalian tissues. The extracellular matrix can be made of small intestinal submucosa (SIS). As known, SIS is a resorbable, acellular, naturally occurring tissue matrix composed of ECM proteins and various growth factors. In other embodiments, the filter portion 20 may be made of a mesh/net cloth; nylon; polymeric material; poly(tetrafluoroethylene), such as Teflon® (DuPont de Nemours); or woven mixtures or combinations thereof.

In use, the device 15 expands from the collapsed state to the expanded state, engaging the struts 25 with the blood vessel 5. In turn, the filter portion 20 expands to capture emboli 49 during treatment of the stenotic lesion. After the device 15 is no longer needed, it may be retrieved. In some embodiments, a catheter is deployed longitudinally about the embolic protection device 15 after it has been collapsed by pulling on the core wire 30 relative to the spiraled section 35.

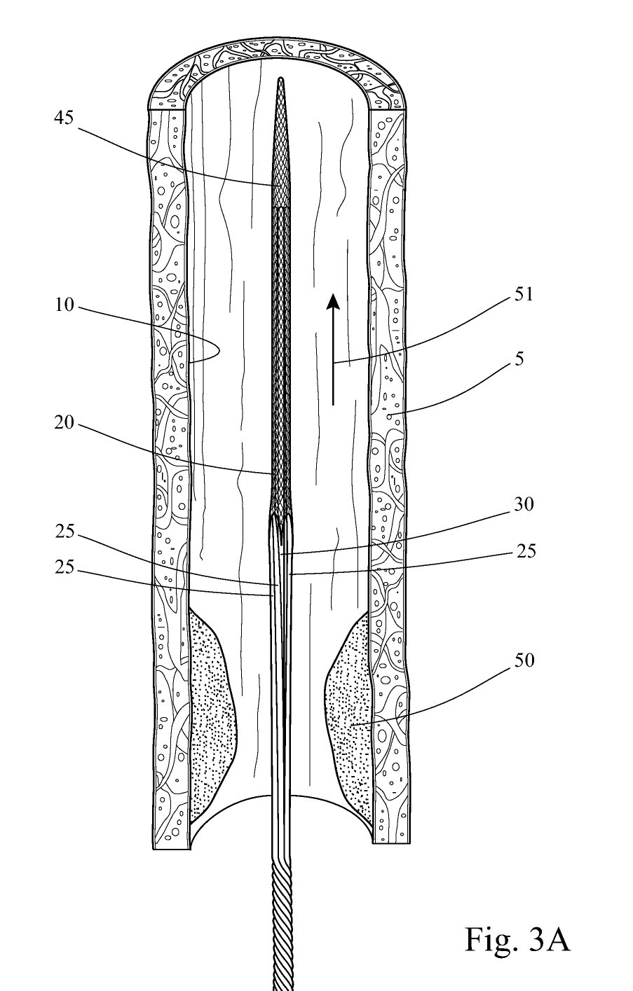

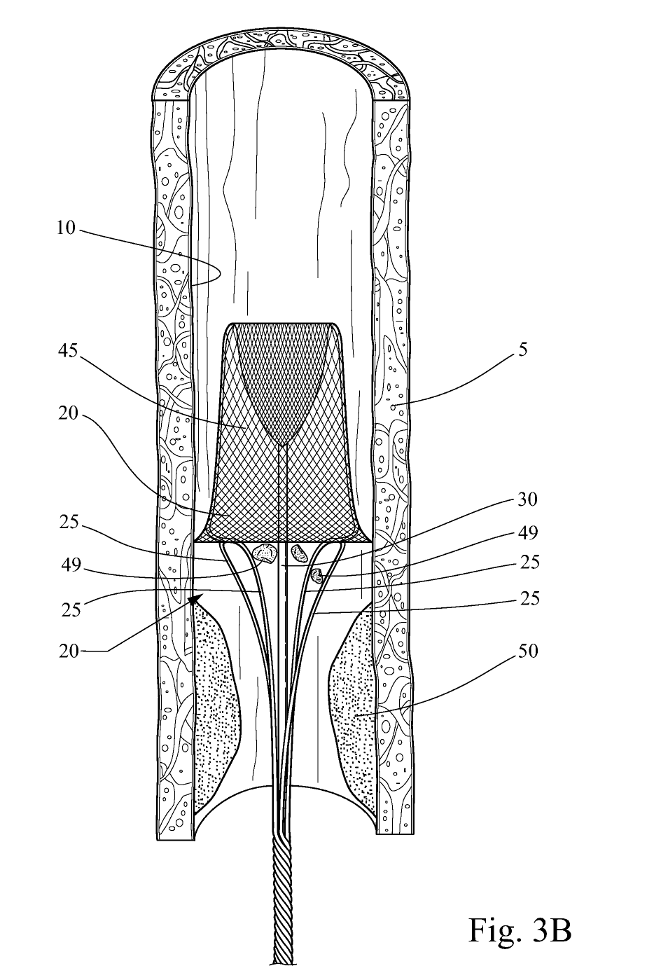

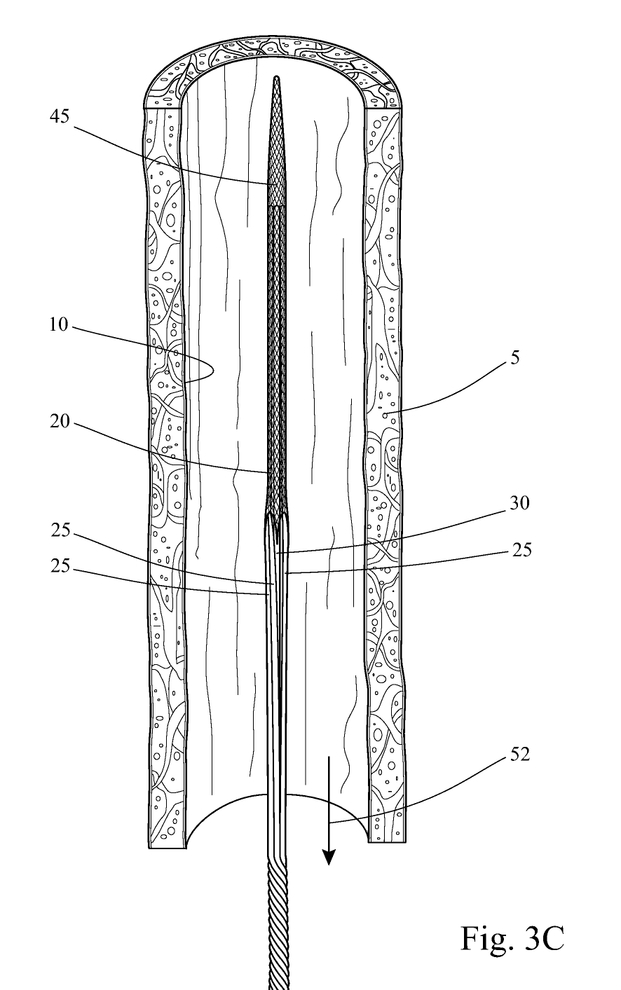

Now referring to FIG. 3A, a cutaway view of a blood vessel 5 is provided illustrating insertion of the embolic protection device 15. The embolic protection device 15 is inserted with the struts 25 in a collapsed state, allowing the device 15 to navigate through the narrow opening formed by the stenosed area 50. Accordingly, during insertion, the profile of the device 15 should be minimized. As such, the core wire 30, which is slideably received by the spiral section 35 of the struts 25 is moved distally relative to the struts 25, thereby drawing the struts 25 and the filter portion 20 tightly against the core wire 30 and forming a collapsed state. The small profile enables the device to pass by a lesion without inadvertently dislodging material from the lesion site. The device 15 is inserted into the vessel 5 past the stenosis 50 as denoted by the distally pointing arrow 51.

Once the struts 25 and filter portion 20 of the embolic protection device 15 is located distal the stenosis 50, the struts 25 can be expanded against the inner wall 10 of the blood vessel 5 as shown in FIG. 3B. In the expanded state, the struts 25 provide a radial force against the filter portion 20 and/or the vessel's inner wall 10, thereby securing the filter portion 20 against the inner wall 10 of the vessel 5. The radial force eliminates gaps between the filter portion 20 and the vessel 5 forcing embolic material 49 that is released from the stenosis 50 to be trapped downstream in the annular chamber 45 of the filter portion 20. After a procedure is performed on the stenosis 50, the core wire 30 is moved distally relative to the struts 25 to collapse the struts 25 and filter portion 20 tightly against the core wire 30, as shown in FIG. 3C. In the collapsed state, the emboli 49 are trapped within the annular chambers 45 of the filter portion 20 and against the core wire 30. However, a catheter may also be slid over the device 15, as a precautionary measure during removal. The device 15 in the collapsed state may then be removed proximally, as denoted by proximally pointing arrow 52.

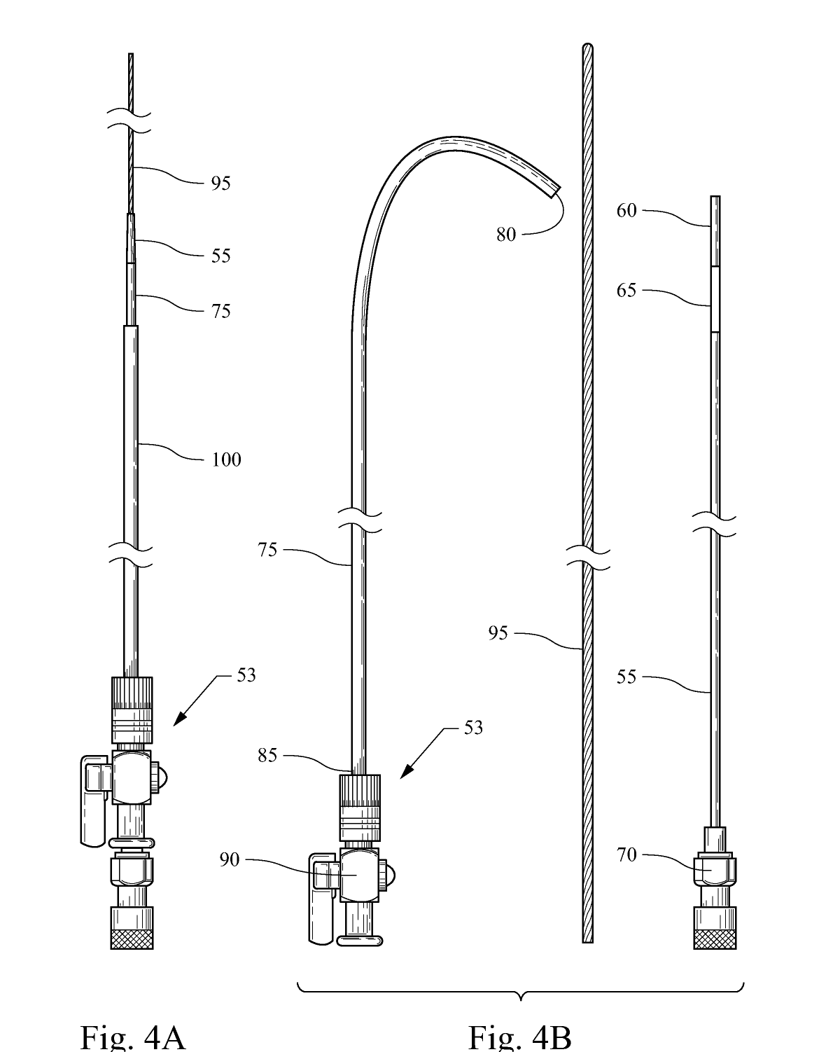

The embolic protection device 15 may be used independently without any other delivery system or mechanism. Alternatively, the device 15 may be used, for example, with an embolic protection assembly 53 as depicted in FIGS. 4A and 4B. As shown, the assembly 53 includes a balloon catheter 55 having a tubular body 60 and an expandable balloon 65 attached to and in communication with the tubular body 60 for angioplasty at a stenotic lesion. The assembly 53 also includes the embolic protection device 15 mentioned above. The tubular body 60 is preferably made of soft flexible material, such as silicone, nylon, or polyurethane, but can be made of any other suitable material. The balloon catheter 55 may include an outer lumen that is in fluid communication with the balloon 65 for inflating and deflating the balloon 65 and an inner lumen formed within the outer lumen for percutaneous guidance through the blood vessel 5 with a wire guide and for deploying the embolic protection device 15. In certain implementations, the balloon catheter 55 has a proximal fluid hub 70 in fluid communication with the balloon 65 by way of the outer lumen for fluid to be passed through the outer lumen for inflation and deflation of the balloon 65 during treatment of the stenotic lesion.

The assembly 53 further includes an inner catheter 75 with a distal end 80 through which the balloon catheter 55 is disposed for deployment in the blood vessel 5. The inner catheter 75 is preferably made of a soft, flexible material such as silicone or any other suitable material. Generally, the inner catheter 75 also has a proximal end 85 and a plastic adaptor or hub 90 to receive the embolic protection device 15 and balloon catheter 55. The size of the inner catheter 75 is based on the size of the body vessel into which the catheter 75 is inserted, and the size of the balloon catheter 55. The assembly 53 may also include a wire guide 95 configured to be percutaneously inserted within the vasculature to guide the inner catheter 75 to a location adjacent a stenotic lesion.

To deploy the embolic protection device 15, the device 15 is placed in the inner lumen of the balloon catheter 55 prior to treatment of the stenotic lesion. The distal protection device is then guided through the inner lumen preferably from the hub 70 and distally beyond the balloon 65 of the balloon catheter 55, exiting from the distal end of the balloon catheter 55 to a location within the vasculature downstream of the stenotic lesion.

The assembly 50 may include a polytetrafluoroethylene (PTFE) introducer sheath 100 for percutaneously introducing the wire guide 95 and the inner catheter 75 in a body vessel. Of course, any other suitable material known to one skilled-in-the-art may be used. The introducer sheath 100 may have any suitable size, e.g., between about three French (0.5 mm) to about seven French (1.3 mm). The introducer sheath 100 serves to allow the inner balloon catheter to be inserted percutaneously to a desired location in the body vessel. The introducer sheath 100 receives the inner catheter 75 and provides stability to the inner catheter at a desired location of the body vessel. For example, as the introducer sheath 100 is held stationary within a common visceral artery, it adds stability to the inner catheter 75, as the inner catheter 75 is advanced through the introducer sheath 100 to a dilatation area in the vasculature.

When the distal end 80 of the inner catheter 75 is at a location downstream of the dilatation area in the body vessel, the balloon catheter 55 is inserted through the inner catheter 75 to the dilatation area. The embolic protection device 15 is then loaded at the proximal end of the balloon catheter 55 and is advanced coaxially through the inner lumen of the balloon catheter 55 for deployment through the distal end of the balloon catheter.

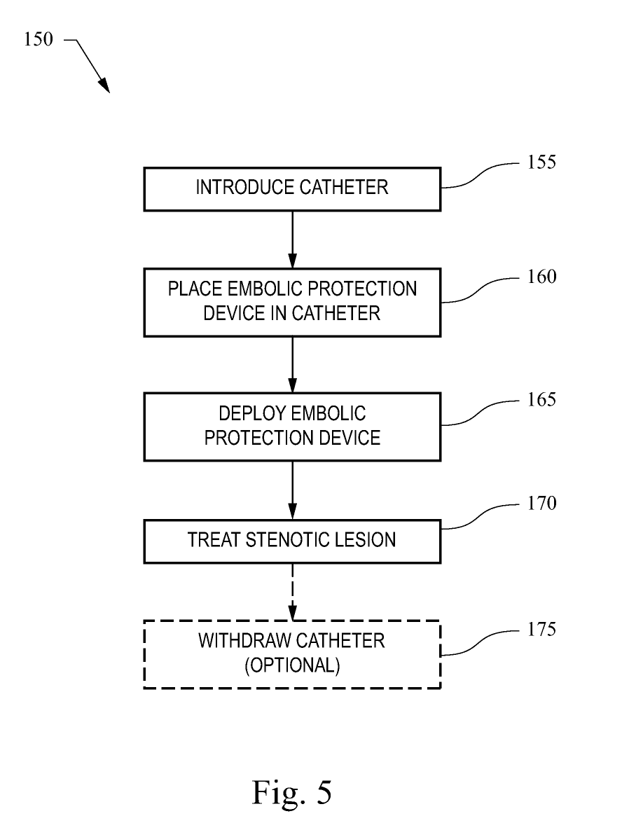

FIG. 5 depicts one method 150 for capturing emboli during treatment of a stenotic lesion in a body vessel, implementing the assembly mentioned above. The method 150 comprises percutaneously introducing a balloon catheter 55 having an expandable balloon 65 for angioplasty of the stenotic lesion in the blood vessel 5 in step 155. Introduction of the balloon catheter 55 may be performed by any suitable means or mechanism. As mentioned above, an introducer sheath 100 and a wire guide 95 may be used to provide support and guidance to the balloon catheter 55. For example, the wire guide 95 may be percutaneously inserted through the introducer sheath 100 to the stenotic lesion in the blood vessel 5. The inner catheter 75 and balloon catheter 55 may then be place over the wire guide 95 for percutaneous guidance and introduction to the stenotic lesion 50. The physician may use any suitable means, for example, fluoroscopy, of verifying the placement of the balloon catheter 55 at a dilatation area.

The method 150 further comprises disposing the embolic protection device 15 coaxially within the balloon catheter 55 in step 160. The device 15 may be disposed coaxially within the balloon catheter 55 before or after percutaneous insertion of the balloon catheter 55. For example, once the balloon catheter 55 is placed at the stenotic lesion 50, the wire guide 95 may be removed therefrom, and the device 15 may then be disposed within the balloon catheter 55 for guidance and introduction in the body vessel 5. In this example, the expandable balloon 65 is positioned at the stenotic lesion 50 and the device 15, in its collapsed state, is disposed through the distal end of the balloon catheter 55 downstream from the expandable balloon 65.

The method 150 further includes deploying the device in a deployed or expanded state downstream from the stenotic lesion 50 to capture emboli during treatment of the stenotic lesion in step 165. In the expanded state, the open end of the filter portion 20 is expanded to a proximally facing concave shape for capturing emboli during angioplasty.

The method 150 may further include treating the stenotic lesion 50 in the blood vessel 5 with the balloon catheter 55 in step 170. In this step, the expandable balloon 65 may be injected with a saline solution, for example, a 50/50 mixture of saline and contrast, and expanded for pre-dilatation. As desired, additional balloon catheters 55 may be used for pre-dilatation treatment, primary dilatation treatment, and post-dilatation treatment of the stenotic lesion while the device is in its expanded state within the body vessel.

Finally, the method 150 may further comprise an optional step 175 in which the catheter is withdrawn. An alternative treatment device may then be placed if desired over the spiraled section 35 of the embolic protection device 15, in other words, the device 15 may serve as a wire guide for any alternative treatment device.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is

1. An embolic protection device for capturing emboli during treatment of a lesion in a blood vessel, the device comprising:
a plurality of struts having a predetermined shape and being configured to move between an expanded state for engagement with the blood vessel and a collapsed state for filter retrieval and delivery; and
a filter portion circumferentially attached to the struts having a proximal end and a distal end; the filter portion extending freely from the proximal end to a closed distal end;
wherein the filter portion forms at least one annulus chamber in the expanded state with the closed distal end of the chamber being not coincident with the center longitudinal axis of the blood vessel;
wherein the filter portion is configured in the expanded state to allow blood to flow there through and to capture emboli in the annulus chamber.

2. The embolic protection device of claim 1, wherein the closed distal end of the annulus chamber is concentric with the longitudinal axis of the blood vessel.

3. The embolic protection device of claim 1, wherein the closed distal end of the annulus chamber is off-center from the longitudinal axis of the blood vessel.

4. The embolic protection device of claim 1, wherein the device further comprises a core wire that is slideably received by a spiral section formed by twisting the proximal ends of the struts.

5. The embolic protection device of claim 1, wherein the annulus chamber is configured to allow passage of blood through the filter portion proximate to the center longitudinal axis of the blood vessel.

6. The embolic protection device of claim 1, wherein the filter portion is made of one selected from the group of cloth, nylon, a polymeric material, poly(tetrafluoroethylene), extracellular matrix (ECM), small intestinal submucosa (SIS), and combinations thereof.

7. The embolic protection device of claim 1, wherein the struts are made of one selected from the group of a superelastic material, shape memory alloy, stainless steel wire, cobalt-chromium-nickel-molybdenum-iron alloy, cobalt-chrome alloy, and nickel-titanium alloy.

8. The embolic protection device of claim 1, wherein the distal ends of the struts are configured to engage the blood vessel to anchor the device thereto.

9. A method for embolic protection during treatment of a stenotic lesion in a blood vessel, the method comprising the steps of:
introducing a catheter into the blood vessel;
placing the embolic protection device in the catheter in a collapsed state;
deploying an embolic protection device in a collapsed state into the blood vessel past the lesion and causing the device to move from the collapsed state to an expanded state in order to capture emboli during treatment, the device comprising:
a plurality of struts having a predetermined shape and being configured to move between an expanded state for engagement with the blood vessel and a collapsed state for filter retrieval and delivery;
a core wire slideably received by a spiral section formed by the struts at their proximal end; and
a filter portion circumferentially attached to the struts having a proximal end and a distal end; the filter portion extending freely from the proximal end to a closed distal end; and forming at least one annulus chamber in the expanded state with the closed distal end being not coincident to the center longitudinal axis of the blood vessel; and
treating the stenotic lesion.

10. The method of claim 9 wherein the step of deploying the embolic protection device further includes a device where the annulus chamber of the filter portion is one selected from the group of being concentric with the center longitudinal axis of the blood vessel and off-center from said longitudinal axis.

11. The method of claim 9, further comprising the step of withdrawing the catheter.

12. The method of claim 9, wherein the step of placing the embolic protection device into the catheter includes moving the core wire relative to the spiral section to close the struts into a collapsed state.

13. The method of claim 9, wherein during the step of deploying the embolic protection device moving from its collapsed state to the expanded state includes expanding the struts against the inner wall of the blood vessel, thereby, providing a radial force against the filter portion that secures the filter portion against the inner wall of the vessel.

14. An assembly for removing emboli from a body vessel, the assembly comprising:
an embolic protection device including a plurality of struts having a predetermined shape and being configured to move between an expanded state for engagement with the body vessel and a collapsed state for filter retrieval and delivery; and a filter portion circumferentially attached to the struts having a proximal end and a distal end; the filter portion extending freely from the proximal end to a closed distal end and forming at least one annulus chamber in the expanded state with the closed distal end of the chamber being not coincident to the center longitudinal axis of the body vessel; and
a balloon catheter having a tubular body portion and an expandable balloon attached to and in fluid communication with the tubular body portion; the balloon catheter facilitating delivery of the embolic protection device in the collapsed state to a position distal to a lesion in the body vessel;
wherein the embolic protection device is configured in the expanded state to allow blood to flow there through and to capture emboli in the annulus chamber of the filter portion.

15. The assembly of claim 14 wherein the balloon catheter includes an outer lumen and an inner lumen, the outer lumen being in fluid communication with the balloon for inflating and deflating the balloon, the inner lumen being formed there through for percutaneous guidance through the body vessel.

16. The assembly of claim 14, wherein the closed distal end of the annulus chamber of the filter portion is concentric to the longitudinal axis of the blood vessel.

17. The assembly of claim 14, wherein the closed distal end of the annulus chamber of the filter portion is off-center from the longitudinal axis of the blood vessel.

18. The assembly of claim 14 further comprising:
an inner catheter having a distal end through which the balloon catheter is disposed for deployment in the body vessel;
a wire guide configured to be disposed through the inner lumen of the balloon catheter for percutaneous guidance through the body vessel; and
an introducer sheath through which the inner catheter is inserted for percutaneous insertion in the body vessel.

19. The assembly of claim 14, wherein the annulus chamber of the embolic protection device is configured to allow passage of more blood flow through the filter portion proximate to the center longitudinal axis of the blood vessel than through the distal portion of the annulus chamber.

20. The assembly of claim 14, wherein the filter portion is made of one selected from the group of cloth, nylon, a polymeric material, poly(tetrafluoroethylene), extracellular matrix (ECM), small intestinal submucosa (SIS), and combinations thereof, while the struts are made of one selected from the group of a superelastic material, shape memory alloy, stainless steel wire, cobalt-chromium-nickel-molybdenum-iron alloy, cobalt-chrome alloy, and nickel-titanium alloy.
